(19) United States
(12) Patent Application Publication
(10) Pub. No.: US 2007/0085063 A1
(43) Pub. Date: Apr. 19, 2007

(54) Tunable sunblock agents

(75) Inventor: Christopher C. Capelli, Houston, TX (US)

Correspondence Address: FULBRIGHT & JAWORSKI L.L.P., 600 CONGRESS AVE., SUITE 2400, AUSTIN, TX, 78701, US

(73) Assignee: The Board Of Regents Of The University Of Texas System

(21) Appl. No.: 11/411,419

(22) Filed: Apr. 26, 2006

Related U.S. Application Data
(60) Provisional application No. 60/719,827, filed on Sep. 23, 2005.
(60) Provisional application No. 60/674,901, filed on Apr. 26, 2005.

(51) Int. Cl. F21V 9/00; A61Q 17/04; A61K 8/18

(52) U.S. Cl. 252/582; 252/588; 106/31.29

(57) ABSTRACT

Compositions for protecting an object from electromagnetic radiation exposure is disclosed. In certain embodiments, the composition can include a plurality of different crystalline colloidal arrays, the arrays comprising particles dispersed within a matrix. At least one of the different crystalline colloidal arrays can randomly orientate within the composition.

### CROSS REFERENCES TO RELATED APPLICATIONS

This application claims the benefit of U.S. Provisional Application No. 60/719,827, filed Sep. 23, 2005 and U.S. Provisional Application No. 60/674,901, filed Apr. 26, 2005. Both provisional applications are incorporated by reference in their entireties.

### BACKGROUND OF THE INVENTION

A. Field of the Invention

The present invention relates generally to sunblock compositions and methods for their use in blocking electromagnetic radiation. In certain aspects, the compositions can diffract electromagnetic radiation over a selected range. The compositions can also allow transmission of electromagnetic radiation over a selected range.

B. Background of the Invention

A common goal of sunblock compositions is to protect the user or article of manufacture from exposure to electromagnetic radiation. Over-exposure of electromagnetic radiation can cause damage to skin, hair, finger nails, and articles of manufacture. For instance, sun-exposure of skin has been shown to cause wrinkles, brown age spots, blotchiness, and leathery, sagging skin. In worst-case scenarios, over-exposure to the sun's electromagnetic radiation can cause skin cancer which can be disfiguring and even deadly.

1. Electromagnetic Radiation

A portion of the electromagnetic spectral distribution emitted by the sun includes wavelengths of electromagnetic energy that range between about 290 and 10000 nanometers (nm). This range can be divided into different regions which include: (1) the ultraviolet (UV) region (290-400 nm); (2) the visible region (400-760 nm) and (3) the near-infrared (IR) region (760-10000 nm).

The UV region is sub-divided into three bands referred to as the UVA, UVB and UVC bands. The UVB band extends from 290 to 320 nm. It is the principal cause of the sunburn reaction. Certain UVB ranges, however, have beneficial aspects. For example, UVB radiation from about 290 to about 315 nm converts the precursor to vitamin D in skin, 7-dehydrocholesterol, to pre-vitamin D₃. Pre-vitamin D₃ subsequently undergoes thermal isomerization to form vitamin D₃ (many humans depend on sun exposure to satisfy their requirements for vitamin D). UVB radiation from about 311-312 has been shown to be effective in the treatment of several types of skin diseases (e.g., psoriasis, atopic dermatitis, seborrheic dermatitis, vitiligo, mycosis fungoides, and other skin diseases).

The UVA band extends from 320-400 nm and is associated with causing the tanning reaction to skin. Although UVA can also cause sunburns, its capacity to do so is less than that of UVB radiation. UVC radiation (200-290 nm) from the sun does not reach the surface of the earth. One can, however, encounter UVC radiation from artificial sources such as germicidal lamps and high and low pressure mercury arc lamps.

IR radiation is sub-divided into three bands referred to as the IRA (760-1400 nm), IRB (1400-3000 nm), and IRC (3000-10000 nm) bands. IR radiation is associated with giving a person a warm feeling when exposed to sunlight. Over-exposure to infrared radiation has been shown to decrease skin elasticity leading to premature aging. Certain IR ranges, however, are beneficial to skin. For example, IR radiation at approximately 890 nm can augment wound healing (Horwitz et al. 1999).

In an effort to solve the problems associated with electromagnetic radiation to skin, several types of sunblock agents have been created (see, e.g., U.S. Pat. Nos. 5,427,771, and 4,828,825).

2. Sunblock Agents

Current topical sunblock agents are typically grouped into two categories: (1) chemical sunblocks; and (2) physical sunblocks. Chemical sunblocks usually include one or more UV-absorbing chemicals. When applied to the surface of skin, these chemicals act as a filter to diminish the penetration of ultra violet radiation to the cells of the epidermis. Physical sunblocks, by contrast, comprise particles of a relatively physiologically inert sunblock. These types of sunblock products are typically messy and occlusive. (Sayre et al, 1990). They tend to form visible, colored (e.g., white) layer on the surface of the skin that can be cosmetically unappealing in many cases.

Recently, new sunblocks have been developed that are relatively transparent. These sunblocks include titanium dioxide or zinc oxide that are “micronized” particles of the metal oxide. While the micronized metal oxides provide a more transparent product, they still suffer from a number of potential problems. For example, a large amount of the micronized metal oxides is needed to achieve adequate sunblock protection. This can be increase the costs associate with preparing such sunblocks. Additionally, the increased quantity can affect the transparency and tactile characteristics of the composition. Studies have also suggested that the regular use of sunblock products can place an individual at risk for vitamin D deficiency and other diseases. (Tangpricha et al. 2004; Holick 2004; Chel et al. 1998).

### SUMMARY OF THE INVENTION

The present invention overcomes the deficiencies in the art by providing compositions and methods for their use in diffracting electromagnetic. In particular non-limiting aspects, the compositions can be used in sunscreen compositions, cosmetic products, and articles of manufacture.

One embodiment of the present invention includes a composition comprising a plurality of different crystalline colloidal arrays. The arrays can include particles dispersed within a matrix. In non-limiting aspects, at least one of the different crystalline colloidal arrays randomly orient in the composition. In other embodiments, at least two, three, four, five, six, seven, or more or all of the different crystalline arrays randomly orient in the composition. The composition can diffract electromagnetic radiation. In certain embodiments, the composition can be designed to diffract and allow transmission of selected electromagnetic radiation.

In one embodiment, the composition includes (a) a first crystalline colloidal array that diffracts electromagnetic radiation over a selected wavelength range; and (b) a second crystalline colloidal array that diffracts electromagnetic radiation over a selected wavelength range that is different than the first colloidal array. The composition, in other non-limiting aspects, can include a third crystalline colloidal array comprising particles dispersed within a third matrix, wherein the third colloidal array diffracts electromagnetic radiation over a selected wavelength range that is different than the first and second colloidal arrays. The composition can even include a fourth crystalline colloidal array comprising particles dispersed within a fourth matrix, wherein the fourth colloidal array diffracts electromagnetic radiation over a selected wavelength range that is different than the first, second, and third colloidal arrays. In certain aspects, the composition can include at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 120, 230, 140, 150, 160, 170, 180, 190, 200, 300, 400, 500, 600, 700, 800, 900, 1000, or more different crystalline colloidal arrays. These different crystalline colloidal arrays can diffract electromagnetic radiation over different wavelength ranges. In non-limiting embodiments, the electromagnetic radiation ranges can overlap and still have different diffraction ranges (e.g., array 1 may diffract electromagnetic radiation over a wavelength range of 100-200 nm while array 2's range is 150-250) or the different arrays can have non-overlapping ranges. In even other aspects, the different crystalline colloidal arrays may have similar or identical diffraction ranges but be different in other aspects as discussed throughout this specification. The compositions of the present invention can, in certain embodiments, includes no more than 5, 6, 7, 8, 9, 10, 11, 12, 13, 14, 15, 20, 25, 30, 35, 40, 45, 50, 55, 60, 65, 70, 75, 80, 85, 90, 95, 100, 110, 120, 130, 140, 150, 1760, 170, 180, 190, 200, 300, 400, 500, 600, 700, 800, 900, 1000, 2000, 3000, 4000, or 5000 different crystalline colloidal arrays. In certain aspects, the compositions include no more than 15 to 100, 20 to 90, 30 to 70, or 15 to 30 different crystalline colloidal arrays.

In still another non-limiting embodiment, the aspect ratio of the crystalline colloidal arrays can have aspect ratios equal to, greater than, or less than about 2:1. For instance, the aspect ratio can be at least about 2:1, 1.9:1, 1.8:1, 1.7:1, 1.6:1, 1.5:1, 1.4:1, 1.3:1, 1.2:1, 1.1:1, 1:1, 0.9:1, 0.8:1, 0.7:1, 0.6:1, or 0.5:1. In other non-limiting examples, the aspect ratio is at least about 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, or more. Arrays having aspect ratios equal to, less than, and/or greater than can be made to randomly orientate in a composition.

In one embodiment, the particles in a crystalline colloidal array can be organized into a periodic array. The periodic array can include a thickness of about 1 to about 50 microns. In other aspects, the periodic array has a thickness of about 1, 2, 3, 4, 5, 6, 7, 8, 9, 10, 11, 12, 13, 14, 16, 16, 17, 18, 19, 20, 21, 22, 23, 24, 25, 26, 27, 28, 29, 30 35, 40, 45, 50, 55, 60, 70, 80 90, 100, or more microns. The periodic array can include about at least 1, 2, 3, 4, 5, 6, 7, 8, 9, 10, 15, 20, 25, 30, 35, 40, 45, 50, 60, 70, 80 90, 100 or more layers of the particles. The distance between the particles in an array can be about 100 to about 1250 nm. In certain aspects, the distance is about 1, 5, 10, 15, 20, 30, 40, 50, 60, 70, 80, 90, 100, 120, 140, 160, 180, 200, 250, 300, 400, 500, 600, 700, 800, 900, 1000, 1100, 1200, 1300, 1400, 1500 or more nanometers. In certain embodiments, the distance between particles is about 100 to about 200 nm or about 300 to about 1250 nm. The particles with the array can also have lattice spacing between each particle. In certain embodiments, different arrays can each have spacing between the particles.

In certain aspects, the particles of the crystalline colloidal arrays can be made up of or include any type of material known to those of ordinary skill in the art. For example, the particles can include an organic polymer or inorganic material. The organic polymer can be, for example, polyurethane, polycarbonate, polystyrene, an acrylic polymer, an alkyd polymer, polyester, siloxane, polysulfide, an epoxy containing polymer, or a polymer derived from an epoxy-containing polymer, or any other organic polymers known to those of skill in the art or disclosed in this specification. The inorganic material can include a metal oxide or a semiconductor or any other inorganic material known to those of skill in the art or disclosed in this specification. For example, the metal oxide can be zinc oxide or titanium dioxide. In other aspects, the particles in a crystalline colloidal array can all be positively or negatively charged. Particles having the same charge can aid in the creation of an ordered pattern. The particles in a crystalline colloidal array can be about the same size or can have different sizes. For example, the particles in one of the crystalline colloidal arrays can differ in size by up to about 1, 2, 3, 4, 5, 6, 7, 8, 9, 10, 11, 12, 13, 14, 15%, or more. In particular embodiments, the particles can have an average size of about 0.01, 0.02, 0.03, 0.04, 0.05, 0.06, 0.07, 0.08, 0.09, 1, 1.1, 1.2, 1.3, 1.4, 1.5, 1.6, 1.7, 1.8, 1.9, 2, 3, 4, or 5 microns. The particles within the matrix can also be fixed in place.

The matrix of the crystalline colloidal array can be made up of or include any type of material known to those of ordinary skill in the art. For example, the matrix can include an organic polymer or inorganic material. In non-limiting aspects, the organic polymer can be polyurethane, polycarbonate, polystyrene, an acrylic polymer, an alkyd polymer, polyester, siloxane, polysulfide, an epoxy containing polymer, or a polymer derived from an epoxy-containing polymer, or any other organic polymer known to those of skill in the art or disclosed in this specification. In certain embodiments, the matrix can be crosslinked. The matrices and particles in the crystalline colloidal arrays can have similar or different refractive indexes. By way of example only, the difference in the refractive indices can be about 0.01, 0.02, 0.03, 0.04, 0.05, 0.06, 0.07, 0.08, 0.09, 0.1, 0.2, 0.3, 0.4, yo about 0.5 or more.

The compositions of the present invention can diffract a broad spectrum of electromagnetic radiation. For example, the compositions can diffract UVA, UVB, UVC, IRA, IRB, and IRC radiation or any combination thereof. For example, a composition can be designed to diffract UVB radiation but not UVA radiation. As discussed throughout this specification, a composition can be designed to diffract and allow a wide range of different electromagnetic ranges (including, for example, ranges within the UVA, UVB, UVC, IRA, IRB, and IRC radiation ranges). By way of example only, the compositions can be designed to diffract electromagnetic radiation having a wavelength of about 200 to about 400, 250 to about 350, 300 to about 325, 200 to about 290, 290 to about 320, or to about 760 to about 2,500 nm. In other aspects, the compositions can permit transmission of a predetermined wavelength range of electromagnetic radiation. Examples of electromagnetic radiation that is not diffracted can include radiation having a wavelength of about 321 to about 400, 290 to about 315, 309 to about 314, or 1660 to about 1900 nm.

The compositions of the present invention can be transparent. The compositions can also be formulated into a sunscreen composition that is applied to skin. The compositions can also be formulated to be spread or sprayed onto the skin. The compositions can be included into a vehicle. The vehicle can include an emulsion, a cream, a lotion, a solution, an anhydrous base, a gel, a spray, or an ointment. The vehicle can be a cosmetic vehicle. The compositions can also be included in a product. The product, in non-limiting embodiments, can be a skin sunscreen product, a skin care product, a sunless skin tanning product, paint, ink, a glass coating, glass, cloth, plastic, or eye glasses, or other products known to those of ordinary skill in the art or identified throughout this specification.

In certain aspects of the present invention, the compositions can include nano scale particles. The nano scale particles can be comprised in the crystalline colloidal arrays in certain embodiments. By way of example only, the nano scale particles can be included within or bound to the matrix and/or the particles of the crystalline colloidal arrays. In other aspects, the nano scale particles are comprised in the composition but not within the arrays. The nano scale particles can be made of or include any material known to those of ordinary skill in the art or identified within this specification. By way of example only, non-limiting materials include metals, metal oxides, metal bromides, semiconductor materials, or an electromagnetic radiation blocking or absorbing chemicals. In certain aspects, the nano scale particles comprise the metal oxide titanium dioxide or zinc oxide or a combination of both. The nano scale particles can be about 1, 5, 10, 15, 20, 25, 30, 35, 40, 45, 50, 60, 70 or more nn in size. In certain aspects, the nano-scale particles are about 10 to about 20 nm in size. In other embodiments, the compositions can include nano scale air bubbles. The nano scale air bubbles can be incorporated into the crystalline colloidal arrays (including the matrices and/or particles) or the composition or both.

In certain aspects, the compositions can include from about 0.1% to about 80% by weight of the crystalline colloidal arrays. In certain aspects, the composition includes from about 1.0% to about 20% or about 1.0% to about 10% by weight of the arrays. As discussed in this specification, the amount of the crystalline colloidal arrays, matrixes, particles, and other ingredients within the composition can be varied to the specific types of electromagnetic radiation blocking compositions desired.

In yet another embodiment of the present invention, there is disclosed a sunscreen composition comprising a plurality of different crystalline colloidal arrays, the arrays comprising particles dispersed within a matrix, wherein the composition is formulated to be applied to skin and diffracts electromagnetic radiation. The sunscreen composition can include a first crystalline colloidal array that diffracts electromagnetic radiation over a selected wavelength range; and a second crystalline colloidal array that diffracts electromagnetic radiation over a selected wavelength range that is different than the first colloidal array. The crystalline colloidal arrays can have an aspect ratio equal, less than, or greater than 2:1. The sunscreen composition can be transparent. The composition can be formulated to be spread or sprayed onto the skin. The sunscreen composition can be included into a vehicle as described throughout this specification. For example, the vehicle can be an emulsion, a cream, a lotion, a solution, an anhydrous base, a gel, a spray, or an ointment. The electromagnetic radiation is UV or IR radiation. The sunscreen composition can be included in a sunscreen product. The sunscreen composition can be comprised in a container. The container can be used to dispense the composition by, for example, spray or squirting the composition. The sunscreen composition can be waterproof. The sunscreen composition can be effective in blocking electromagnetic radiation for at least 2, 3, 4, 5, 6, 7, 8, 9, or 10 hours. The sunscreen composition can be a cream, a lotion, a solution, an anhydrous base, a gel, a spray, or an ointment.

Also disclosed in the present invention is a method of protecting an object from electromagnetic radiation comprising applying on the surface of the object or in incorporating into the object the compositions of the present invention. The composition can be topically applied to the object. The object can be skin, hair, or fingernails (including human and animal skin, hair, or fingernails). In certain aspects, the composition can be formulated for application at least once, twice, three, four, five or more times a day to the skin. In other aspects, the composition is sprayed, spread, or rubbed onto the object. The composition in certain embodiments, can be incorporated into the object. The object, by way of example only, can be any article of manufacture known to those of skill in the art or identified in this specification. For example, the object can be paint, ink, windows, self adhesive tap, eye wear (including eye glasses and contact), cloths (including clothing, car covers, boat covers), wood, protective coatings (e.g., water sealers, stains, ext.) or plastics.

Another aspects of the present invention discloses a method of making a composition comprising a comprising a plurality of different crystalline colloidal arrays, the method comprising (i) obtaining a plurality of different crystalline colloidal arrays; (ii) obtaining a vehicle; and (iii) admixing (i) and (ii), wherein the admixture is formulated into a composition. Non-limiting examples of vehicles contemplated as being useful with the present invention include those identified in this specification or known to those of skill in the art. For example, the vehicle can include an emulsion (e.g., water-in-oil, or oil-in-water), a cream, a lotion, a solution, an anhydrous base, a gel, a spray, or an ointment. In other aspects, the composition can be formulated into a liquid, a spray, an aerosol, or a dry powder. The method can further include randomly orienting the plurality of different crystalline colloidal arrays in the composition. The arrays can have can have an aspect ratio equal to, less than, or greater than 2:1. The composition can be formulated to diffract and allow transmission of selected ranges of electromagnetic radiation.

Also disclosed is a kit comprising the compositions of the present invention. The compositions can be included in a container. In non-limiting aspects, the container can be a bottle, a dispenser, or a package. In certain embodiments, the container can dispense a pre-determined amount of the composition. The composition can be dispensed in a spray, an aerosol, or in a liquid form or semi-solid form. In certain aspects, the container can include indicia on its surface. The indicia, for example, can be a word, a phrase, an abbreviation, a picture, or a symbol. The word or phrase can be “sunscreen,” “sunblock,” “UV specific sunblock,” ext.

In another embodiment, there is disclosed a product or article of manufacture comprising the compositions of the present invention. Product and articles of manufacture that are contemplated as being useful with the present invention are those known to a person of ordinary skill in the art and those identified in this specification. Non-limiting examples include sunscreen products, sunblock products, cosmetic products (e.g., sunless tanning product, moisturizers, creams, lotions, skin softeners, foundations, night creams, lipsticks, cleansers, toners, masks, and other make-up products), paint, ink, cloths (e.g., clothing, tarps, car and boat covers, ext.), glass, glass films, eye ware (e.g., eye glasses and contacts), coatings, windows, plastics, ext.

“Aspect ratio” as used in this specification includes taking the ratio for the longest planar dimension of the outer surface of a crystalline colloidal array to the edge thickness of the array.

“Flakes” include particles of all shapes and sizes.

“Particles” can have a multiple of different shapes, including, but not limited to, spheres, ovals, squares, or any type of irregular shape.

“Sunblock” compositions include compositions that can block electromagnetic radiation from transmitting to skin.

“Blocking” refers to protecting from, diffracting, or other means to keep electromagnetic radiation from transmitting through the composition.

It is contemplated that any embodiment discussed in this specification can be implemented with respect to any method or composition of the invention, and vice versa. Furthermore, compositions of the invention can be used to achieve methods of the invention.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

FIG. 1: Illustrates the blockage of electromagnetic radiation by a crystalline colloidal array.

FIG. 2A and FIG. 2B: (A) Illustrates a UV sunblock composition having crystalline colloidal arrays that self orientate within the composition. (B) The composition diffracts UV radiation from about 210 to about 250 nm.

FIG. 3A and FIG. 3B: (A) Illustrates a UV sunblock composition having crystalline colloidal arrays that randomly orientate within the composition. (B) The composition diffracts UV radiation from about 190 to about 350 nm.

FIG. 4A and FIG. 4B: (A) Illustrates a UV sunblock composition that has five different crystalline colloidal arrays that randomly orientate within the composition. (B) The composition diffracts UV radiation from about 140 to about 400 nm.

FIG. 5A and FIG. 5B: (A) Illustrates a UV sunblock composition that has four different crystalline colloidal arrays that randomly orientate within the composition. (B) The composition diffracts UV radiation from about 140 to about 290 nm and from about 315 to about 400 m but allows transmission of UVB radiation from about 290 to about 315 nm.

FIG. 6A and FIG. 6B: (A) Illustrates a UV sunblock composition that has five different crystalline colloidal arrays that randomly orientate within the composition. (B) The composition is capable of blocking UV radiation over a broad bandwidth of radiation but tuned to allow variable transmission of UV radiation.

FIG. 7A and FIG. 7B: (A) Illustrates an IR sunblock composition having crystalline colloidal arrays that self orientate within the composition. (B) The composition diffracts IR radiation from about 1240 to about 1520 nm.

FIG. 8A and FIG. 8B: (A) Illustrates an IR sunblock composition having crystalline colloidal arrays that randomly orientate within the composition. (B) The composition diffracts IR radiation from about 1100 to about 2220 nm.

FIG. 9A and FIG. 91B: (A) Illustrates an IR sunblock composition that has five different crystalline colloidal arrays that randomly orientate within the composition. (B) The composition diffracts IR radiation from about 750 to about 2570 nm.

FIG. 10A and FIG. 10B: (A) Illustrates an IR sunblock composition that has four different crystalline colloidal arrays that randomly orientate within the composition. (B) The composition diffracts IR radiation from about 750 to about 1660 nm and from about 1900 to about 2570 nm but allows transmission of IRB radiation from about 1660 to about 1900 nm.

FIG. 11A and FIG. 11B: (A) Illustrates an IR sunblock composition that has five different crystalline colloidal arrays that randomly orientate within the composition. (B) The composition is capable of blocking IR radiation over a broad bandwidth of radiation but tuned to allow variable transmission of IR radiation.

### DESCRIPTION OF ILLUSTRATIVE EMBODIMENTS

The use of sunblock compositions have gained more and more popularity over the years. For example, sunblock compositions can be used to protect a person's skin, hair, finger nails, or an article of manufacture from the sun's or artificial electromagnetic radiation. A problem associated with previous sunblock compositions, however, is their inability to allow transmission of selective electromagnetic radiation to a person's skin. Additionally, skin types vary widely among individuals which can affect the efficacy of a given sunblock composition (i.e., a given composition may work well for one individual but not another due to different in skin types).

The inventor has discovered a composition that has several advantages over previous compositions. The compositions, in non-limiting aspects, include a plurality of different crystalline colloidal arrays that can diffract electromagnetic radiation. The compositions can be used to protect, for example, a person's skin, hair, finger nails, or an article of manufacture from damaging electromagnetic radiation such as UV or IR radiation. The arrays include particles that are dispersed within a matrix. The arrays can randomly orientate in the composition. These characteristics, for example, can allow for the production of a composition that blocks and allows transmission of electromagnetic radiation over selective ranges.

These and other aspects of the present invention are described in further detail in the following sections.

A. Crystalline Colloidal Arrays

The crystalline colloidal arrays of the present invention are capable of diffracting electromagnetic radiation. In non-limiting aspects, the arrays include particles dispersed within a matrix. A description of a non-limiting crystalline colloidal array of the present invention, the types of particles and matrices that can be used, and methods of making arrays, are described in the following subsections.

1. Description of a Crystalline Colloidal Array

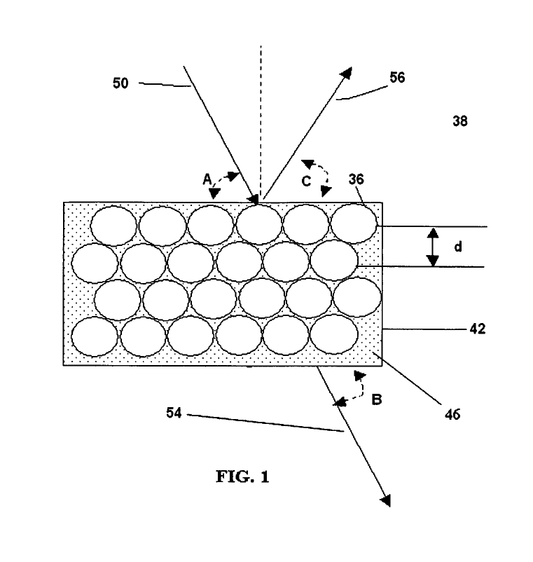

FIG. 1 provides a non-limiting description of a crystalline colloidal array of the present invention. A beam 50 of electromagnetic radiation that includes a full spectrum of visible light, UV, and IR radiation is incident upon a crystalline colloidal array material 38 at an angle A. The lattice spacing between each particle 36 that make up the crystalline colloidal array 38 is in the range of 100-200 nm. The diffracted UV radiation band beam 56 satisfies the Bragg diffraction equation:
mλ.=2nd sin A
where m is an integer (m=1, 2, 3, 4, 5, 6, 7, 8, 9, 10, 11, 12, 13, 14, 15, 16, 17, 18, 19, 20 . . . ) which will preferably be about 1; n is the effective refractive index of the crystalline colloidal array material 38 ; “A” is angle A in FIG. 1, and λ represents wavelength. “d” represents the distance between the layers of particles that make up the crystalline colloidal array material within the solid structure. An effective refractive index (n) is closely approximated as a volume average of the refractive index of the particles (referred to as RI_{particles}) and the refractive index of the polymer matrix 42 (referred to as RIₘₐₜᵣᵢₓ) present in the crystalline colloidal array material determined according to the equation:
n=(vol. % particles/100)×RI_{particles}+(vol. % polymer/100)×RIₘₐₜᵣᵢₓ

The transmitted beam 54 departs the crystalline colloidal array material 38 at angle to B which is substantially equal to angle A. UV radiation beam 56 is Bragg diffracted from the crystalline colloidal array material 38 at an angle C. In this manner, the UV radiation wavelength band beam 56 is effectively filtered from electromagnetic radiation beam 50.

In non-limiting aspects, the wavelength and intensity of the reflected UV radiation beam 56 can be selected by varying the spacing (d) between the particles 36 (i.e, by adjusting the size of the particles), the number of particle layers, the difference in the refractive index between the polymeric matrix 42 and the particles 36, and/or the effective refractive index (n) of the crystalline colloidal array material 38.

When the refractive index of the particles 36 (RI_{particles}) is close to the refractive index of the polymer matrix 42 (RIₘₐₜᵣᵢₓ), the polymer matrix 42 composition may be adjusted to sufficiently change RIₘₐₜᵣᵢₓ to increase the difference between RI_{particles} and RIₘₐₜᵣᵢₓ. This may be accomplished by adding nanoscale particles 46 (sized about 1 to about 50 mn) to the matrix 42 . The nanoscale particles 46 can have particle sizes less than the wavelength of visible light and, thus, do not substantially reflect or scatter light. In non-limiting aspects, suitable materials for the nanoscale particles 46 that increase the effective RIₘₐₜᵣᵢₓ include metals (e.g., gold, silver, platinum, copper, titanium, zinc, nickel), metal oxides (e.g., aluminum oxide, cerium oxide, zinc oxide, titanium dioxide), mixed metal oxides, metal bromides, and semiconductors. Non-limiting materials for nanoscale particles 46 that decrease the effective RIₘₐₜᵣᵢₓ include metal oxides (e.g., silica), mixed metal oxides, and metal fluorides (e.g., magnesium fluoride and calcium fluoride). The RI_{particles} may be adjusted by adding nanoscale particles 46 to or within the particles 36 . Preferred nanoscale particles 46 include titanium dioxide, zinc oxide or mixtures of the two. Nanoscale air bubbles may also be produced in the polymer matrix 42 to decrease RIₘₐₜᵣᵢₓ.

In other non-limiting aspects, preferred crystalline colloidal arrays 38 include an ordered periodic array of particles 36 held in a matrix 42 wherein the difference in refractive index between the matrix and the particles is at least about 0.01, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or more, preferably at least about 0.05, and, more preferably, at least about 0.1. The array of particles 36 can be greater than several millimeters thick (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more microns thick).

The particles 36, in certain aspects, have substantially the same size. In certain other aspects, the particles 36 may differ in size by about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100% or more, preferably by about 5 to 15%. The average particle size is about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4, 5, 6, 7, 8, 9, 10, or more microns, preferably about 0.01 to about 1 micron, and more preferably about 0.06 to about 0.5 microns. The distance d between the particle layers can be controlled by the size of the particles 36 . In certain aspects, the surface of each particle 36 contacts at least one other particle. In other embodiments, the surface of the particles 36 do not contact any other particle. A distribution in particle size causes variation in the wavelength of diffracted electromagnetic radiation. This can be used to make designer sunblocks (e.g., sunscreen for a specific skin type, broadband sunblocks (including sunscreens), compositions that block and allow transmission of specific electromagnetic radiation, etc.).

The array 38 preferably includes 4 layers of particles 36 . It is contemplated that the array, in other embodiments, can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more layers of particles 36 . Non-limiting examples of the types of particles 36 that can be used with the present invention are described in U.S. Pat. Nos. 5,944,994 and 6,894,086. Examples include particles 36 comprising an organic polymer (e.g., polyurethane, polycarbonate, polystyrene, an acrylic polymer, an alkyd polymer, polyester, siloxane polymer, polysulfide, an epoxy-containing polymer or a polymer derived from an epoxy-containing polymer. Other examples include particles comprising an inorganic polymer, such as a metal oxide (e.g., alumina, silica or titanium dioxide) or a semiconductor (e.g., cadmium selenide). In other aspects, the particles are cross-linked. The material chosen depends upon the optimum degree of ordering desired in the resulting lattice. In certain embodiments, the particles preferably include zinc oxide or titanium dioxide.

The matrix 42 can include a variety of materials known to those of ordinary skill in the art. For example, U.S. Pat. Nos. 5,944,994 and 6,894,086 provide a number of non-limiting matrices that can be used with the present invention. In certain aspects, for example, the matrix 42 includes a polymeric composition. The polymeric composition can be a curable polymeric composition such as a UV curable composition with high acrylate content. Non-limiting examples of polymers for the matrix 42 include polyurethanes, acrylic polymers, alkyd polymers, polyesters, siloxane-containing polymers, polysulfides, epoxy-containing polymers, and polymers derived from epoxy-containing polymers. In certain aspects, the matrix 42 can include at least 1, 2, 3, 4, 5, 6, 7, 8, 9, 10, 11, 12, 13, 14, 15, 16, 17, 18, 19, 20, or more different polymer materials.

2. Methods of Preparing Crystalline Colloidal Arrays

Methods and corresponding examples of how to make crystalline colloidal arrays are explained in U.S. Pat. Nos. 5,944,994 and 6,894,086, both of which are incorporated into this application by reference. By way of example only, and with reference to U.S. Pat. No. 5,944,994, particles of the present invention are placed into a liquid medium. The medium, in non-limiting aspects, can be water, glycerol, ethylene glycol, methanol, ethanol, dimethyl sulfoxide, phenyl methyl sulfoxide, dioxane, dimethylformamide, polyethylene glycol, or glycerine, or any material possessing similar properties. The particles and medium can be placed into a sealed chamber. The chamber, in preferred aspects, is made of quartz, LEXAN or LEXAN-coated glass. The suspension that includes the particles and medium is then diluted with deionized, doubly distilled water to provide a partial volume fraction in the range of about 0.5 to 75 percent. The sealed chamber is subsequently placed in room temperature water for a period of time adequate to allow the array to crystallize. This environment should also be perturbation-free. Geometric ordering of the crystalline structure can then occurs.

A solvent (e.g., benzene, toluene, chloroform, ext.) is then added to a polymer latex solution. This solution is added to the medium to fuse the particles together, thereby creating an ordered array. The medium is subsequently removed by gentle evaporation at a temperature between about 20 to 30° C. until the desired evaporation takes place. The evaporation condenses the particles into a three-dimensional array having highly periodic lattice spacing. This lattice spacing is created in a manner such that it can diffract a predetermined wavelength band. The resulting crystalline colloidal array is then removed from the chamber. The EM radiation diffraction range is dependent on the lattice structure. One method of fixing the particles in the desired relative position involves polymerization of the medium surrounding the particles. For example, polymerization can be performed by adding acrylamide or bisacrylamide and preferably a nonionic UV photoinitiator to a colloidal solution contained between two quartz plates. Ultraviolet light is then utilized to initiate the polymerization.

In other embodiments, the particles are fixed in the polymeric matrix by providing a dispersion of the particles, bearing a similar charge, in a carrier, applying the dispersion onto a substrate, evaporating the carrier to produce an ordered periodic array of the particles on the substrate, coating the array of particles with the polymer, and curing the polymer to fix the array of particles within the polymer. The dispersion may contain about 1 to about 70 vol. % of the charged particles, preferably about 30 to about 65 vol. % of the charged particles. The fixed array is removed from the substrate and converted into particulate form. The substrate may be a flexible material (such as a polyester film) or an inflexible material (such as glass). The dispersion can be applied to the substrate by dipping, spraying, brushing, roll coating, curtain coating, flow coating or die coating to a desired thickness (e.g. a thickness of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more microns), preferably a maximum thickness of about 20 microns, more preferably a maximum of about 10 microns, most preferably a maximum of about 5 microns. The fixed array of particles is removed from the substrate in the form of an extended film or in the form of flakes that may be suspended in a coating composition.

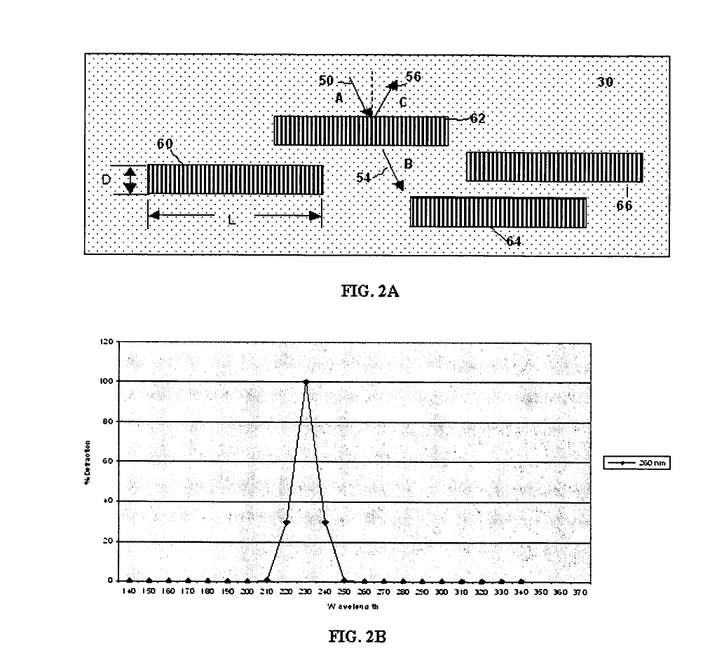

Further, and as noted above, the wavelength and intensity of the reflected electromagnetic radiation beam (See FIG. 1) can be selected by varying the spacing (d) between the particles 36 (i.e, by adjusting the size of the particles), the number of particle layers, the difference in the refractive index between the polymeric matrix 42 and the particles 36, and/or the effective refractive index (n) of the crystalline colloidal array material 38. Therefore, different crystalline colloidal arrays that are designed to block a specific range of electromagnetic radiation (See FIGS. 2-11) (all ranges of electromagnetic radiation are contemplated by the inventor (e.g. ranges within or the entire range of UVA, UVB, UVC, IRA, IRB, and IRC radiation) can be made. Additionally, a person of ordinary skill in the art can determine the diffraction range of a given crystalline colloidal array without undue experimentation. For example, the electromagnetic diffraction range/capabilities of an array can be determined by SPF determination tests, or by calculating the UV or IR efficiency values (see, e.g., U.S. Pat. No. 6,290,938 and U.S. Sunscreen Tentative Final Monograph, issued in May, 1993).

B. Sunblock Compositions

Formulating sunblock and sunscreen compositions are known to those of ordinary skill in the art. For example, a sunblock formulation is described in U.S. Pat. No. 6,894,086, which is incorporated by reference.

The crystalline colloidal arrays of the present invention can be used to prepare many different types of sunblock compositions. For example, the arrays can be used to design sunscreen compositions for a particular skin type (e.g., fair, medium, or dark skin, or skin that tan's quickly or slowly). Other non-limiting examples include broadband sunblock compositions, sunscreen compositions (e.g., compositions that allow UVA radiation but block UVB radiation, compositions that block IR radiation, sunscreen compositions that block UV and IR radiation, therapeutic sunscreen compositions that allow the skin to be exposed to beneficial electromagnetic radiation, and other compositions disclosed throughout this composition).

In certain aspects, for example, the compositions or crystalline colloidal arrays of the present invention can be designed to diffract and/or allow transmission of electromagnetic radiation ranging from 290 to 10000 nm, and any number derivable therein (e.g., 291, 292, 293, 294, 295, 296, 297, 298, 299, 300, 301, 302, 303, 304, 305, 306, 307, 308, 309, 310, 311, 312, 313, 314, 315, 516, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 412, 420, 421, . . . 9,990, 9,991, 9,992, 9,993, 9,994, 9,995, 9,996, 9,997, 9,998, 9,999).

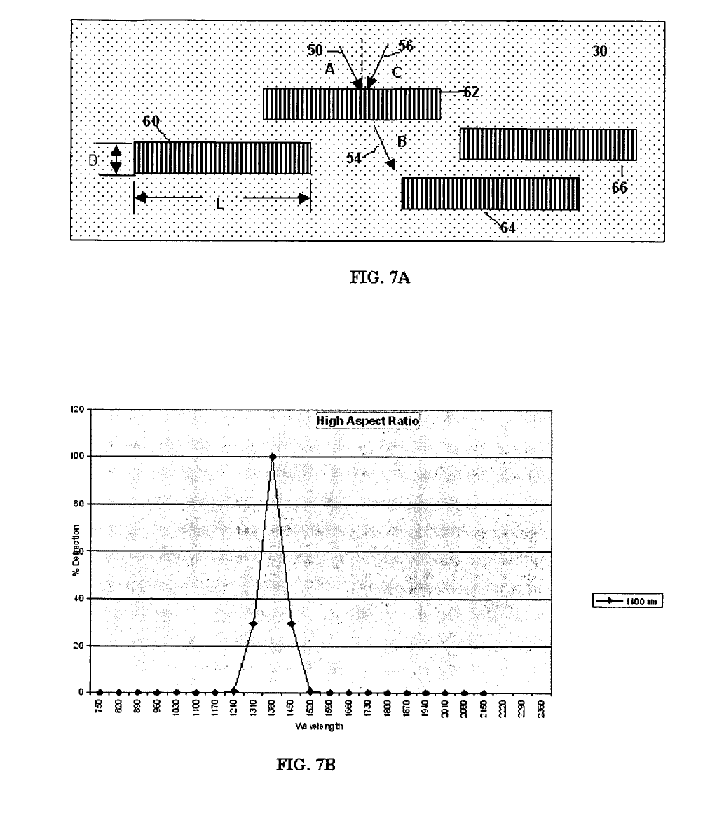

FIGS. 2 and 7, for example, are illustrations compositions that diffract a single narrowband of UV and IR radiation, respectively. Referring to FIG. 2A, the composition includes four identical crystalline colloidal arrays 60, 62, 64, 66. The arrays 60, 62, 64, 66 have an aspect ratio that is greater than 2:1 and self orientate. When UV light 50 is radiated on the composition, only a narrow band UV radiation 56 is diffracted. FIG. 2B illustrates the percentage UV radiation diffracted. For this example, UV light at 230 nm that is incident on the composition at 90 degrees is essentially all diffracted. However, the curve is narrow thereby showing that the composition 10 only blocks a narrow range of UV radiation (i.e., 210-250 nm). FIGS. 7A and 7B provide similar illustrations for an IR blocking composition. In order to create a broadband sunblock composition with arrays that self orientate, a larger number of different crystalline colloidal arrays would have to be used.

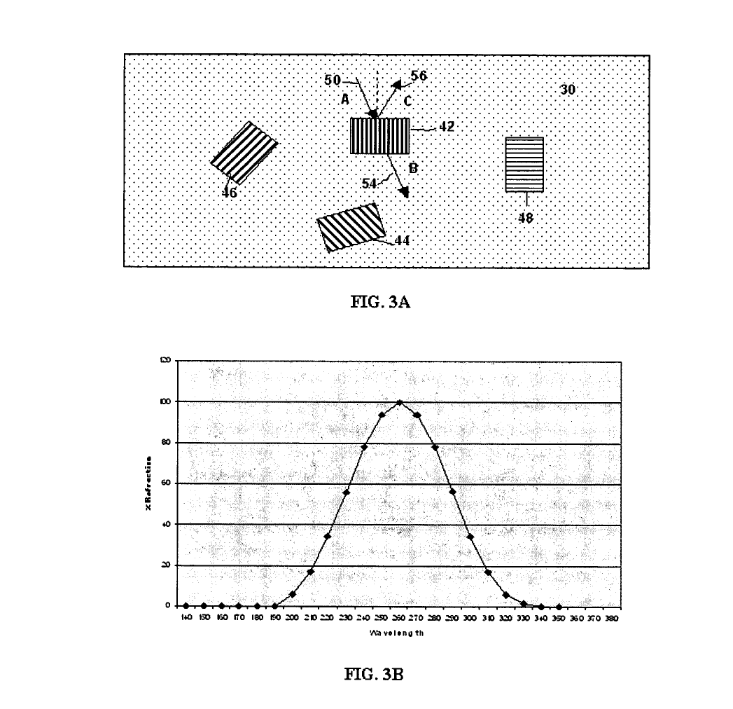

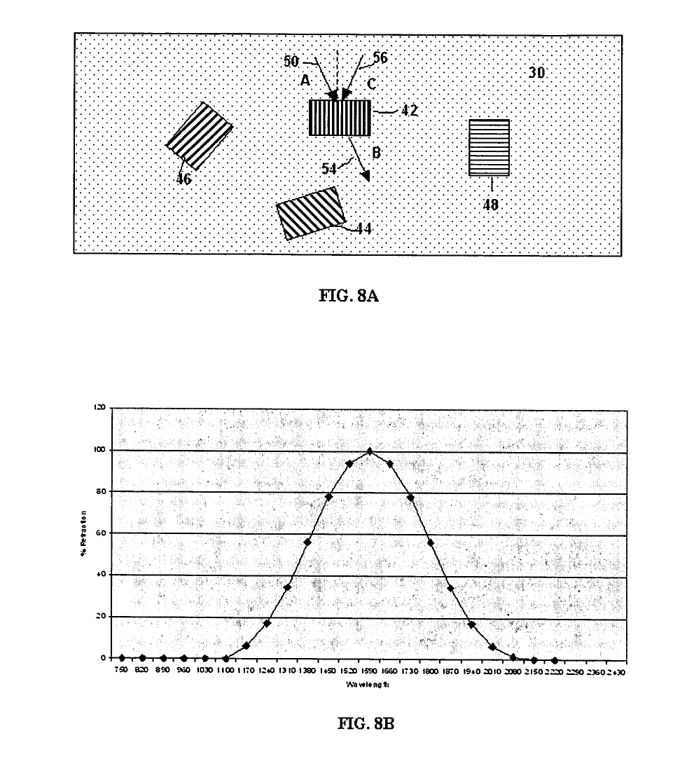

FIGS. 3 and 8 are illustrations of compositions that diffract a single broader band of UV and IR radiation, respectively. Referring to FIG. 3A, the composition includes four identical crystalline colloidal arrays 44, 46, 48, 54 that are designed to diffract UV radiation at centered at 260 nm. The arrays 20 have a low aspect ratio and randomly orientate within the composition 10 . This provides for a UV diffraction range from about 190 to about 350 nm. Given the random orientation of each array 44, 46, 48, 54, the angle of incident electromagnetic radiation (i.e., angle A) on each array 44, 46, 48, 54 will be random. As a result, the wavelength of electromagnetic radiation that is diffracted for each array 44, 46, 48, 54 will vary. This causes the composition to have poor diffraction efficiency over a certain UV band range and an increased diffraction over a selected UV Band range. FIGS. 8A and 8B provide similar illustrations for an IR blocking composition (e.g., the crystalline colloidal arrays 44, 46, 48, 54 are designed to diffract IR radiation centered at 1530 nm).

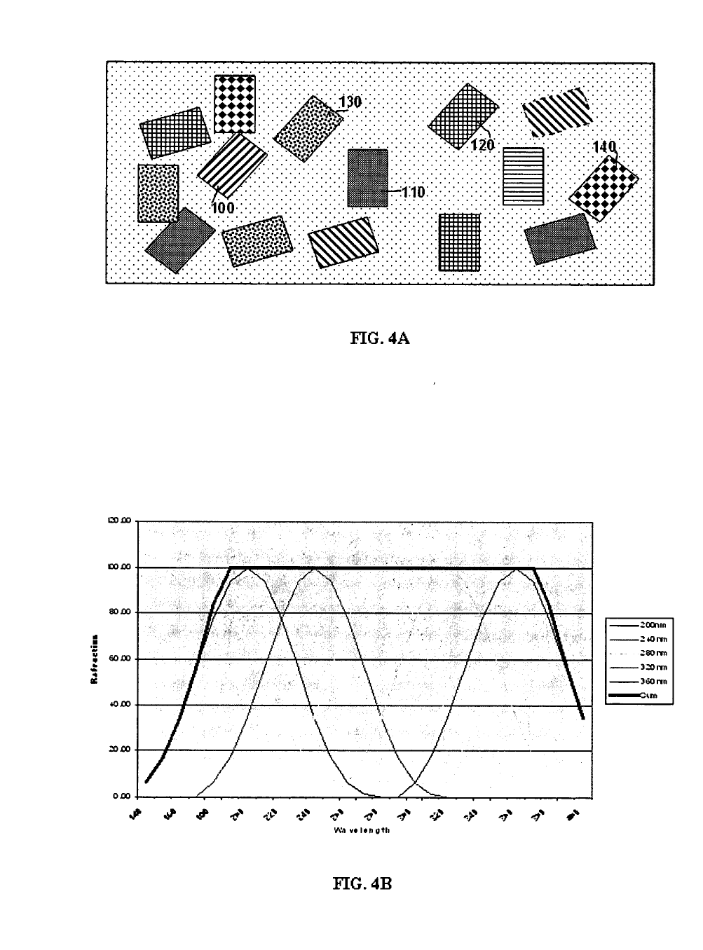

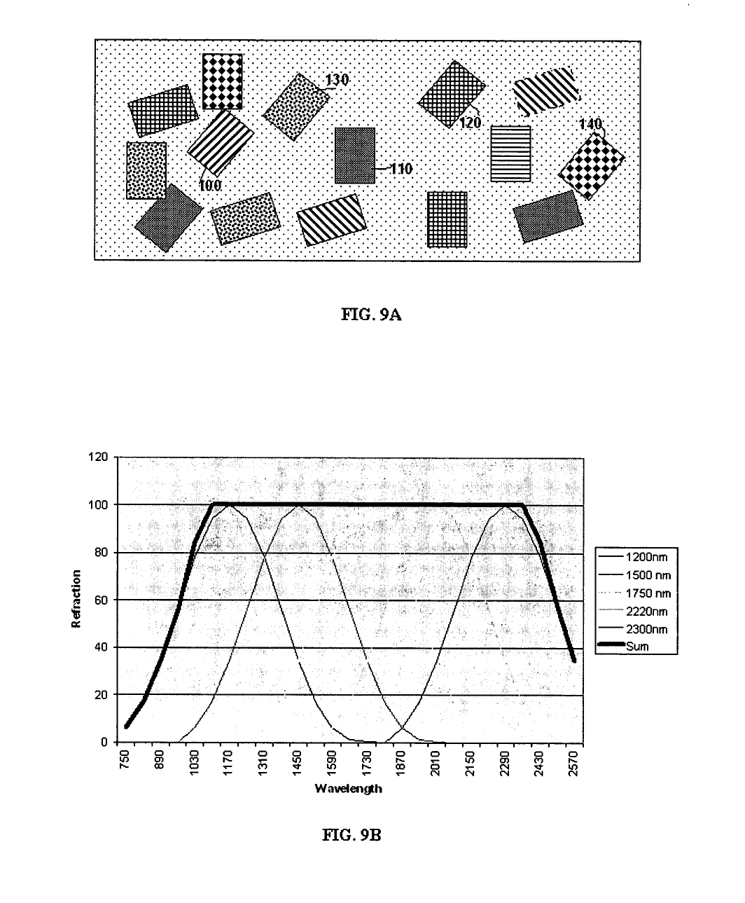

FIGS. 4 and 9 are illustrations of UV and IR blocking compositions that have five different types of crystalline colloidal arrays respectively. Referring to FIG. 4A, the five different crystalline colloidal array materials 100, 110, 120, 130, 140 are designed to diffract UV radiation centered at approximately 200 nm, 240 nm, 280 nm, 320 nm and 360 nm respectively. Each of the five arrays 100, 110, 120, 130, 140 have a low aspect ratio and randomly orientate within the composition. This produces a relatively broad spectrum of UV blockage centered on each crystalline colloidal array material's specific wavelength. The net effect, as shown in FIG. 4B, is a composition that has broad UVA/UVB protection from about 140 to about 400 nm. FIGS. 9A and 9B provide similar illustrations for an IR blocking composition (e.g., there are five different crystalline colloidal arrays 100, 110, 120, 130, 140 that are designed to diffract IR radiation centered at approximately 1200 nm, 1500 nm, 1750 nm, 2220 nm, and 2300 nm, respectively).

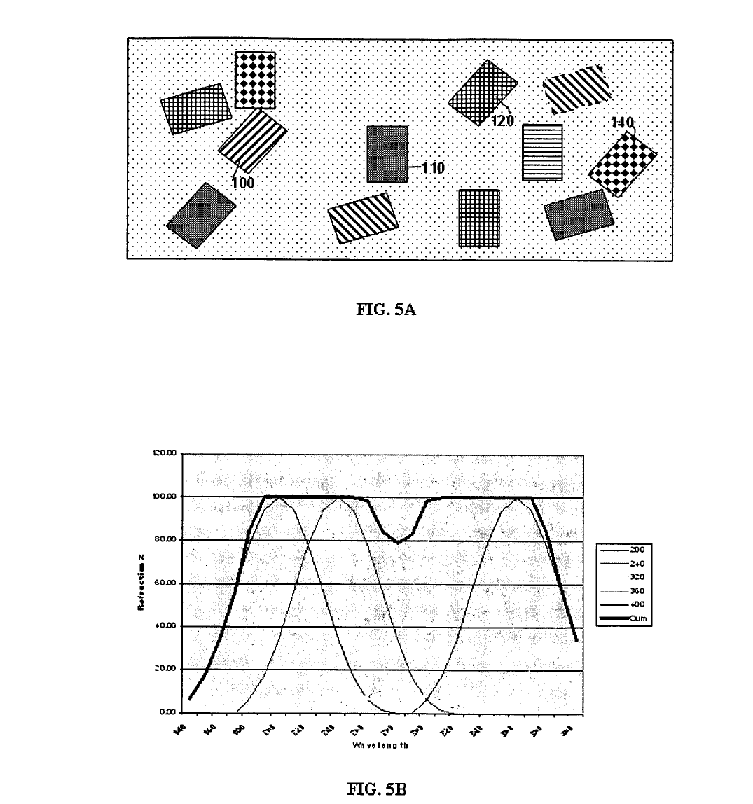

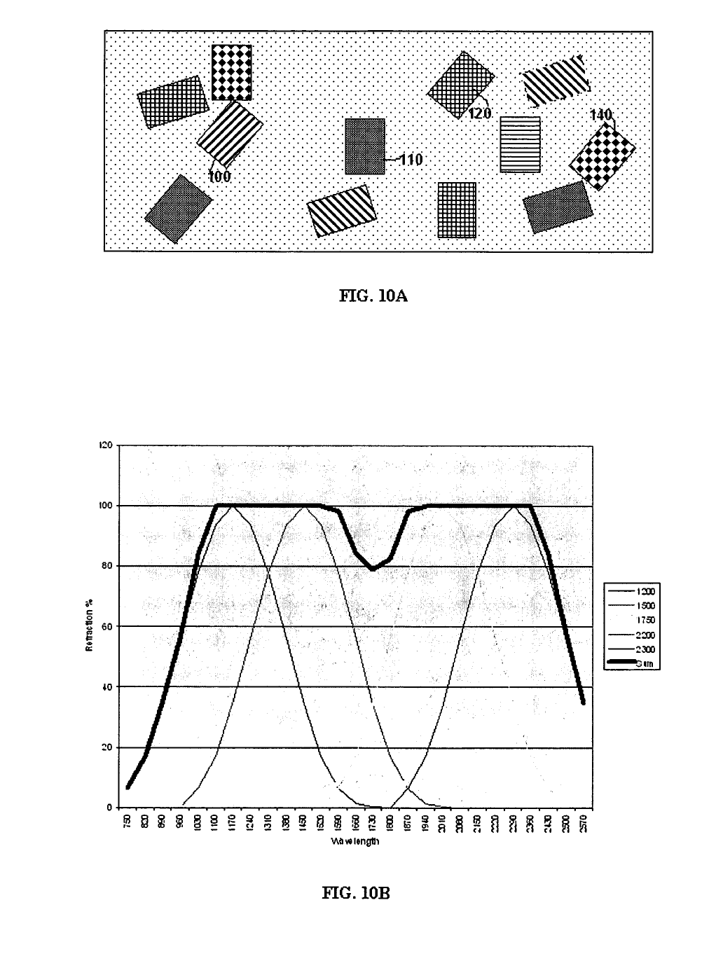

FIGS. 5 and 10 are illustrations of UV and IR blocking compositions that have four different types of crystalline colloidal arrays respectively. Referring to FIG. 5A, the four different arrays 100, 110, 120, 140 are designed to diffract UV radiation centered at approximately 200 nm, 240 nm, 329 nm and 360 nm, respectively. The net effect, as shown in FIG. 5B, is a composition that has broad UVA/UVB protection while allowing transmission of UVB radiation in the bandwidth of 290-315 nm. In other words, the crystalline colloidal array designed to block UV radiation in the bandwidth of 290-315 nm is omitted (or minimized in the final composition for the sunblock product). In a non-limiting aspect, the composition 10 is useful for individuals who need sunblock protection, but are at risk of Vitamin D deficiency. FIGS. 10A and 10B provide similar illustrations for an IR blocking composition (e.g., there are four different crystalline colloidal arrays 100, 110, 120, 140 that are designed to diffract IR radiation centered at approximately 1200 nm, 1500 nm, 220 nm, 2220 nm, and 2300 nm, respectively). FIG. 10B illustrates a composition that diffracts IR radiation from about 750 to about 1660 nm and from about 1900 to about 2570 nm but allows transmission of IRB radiation from about 1660 to about 1900 nm.

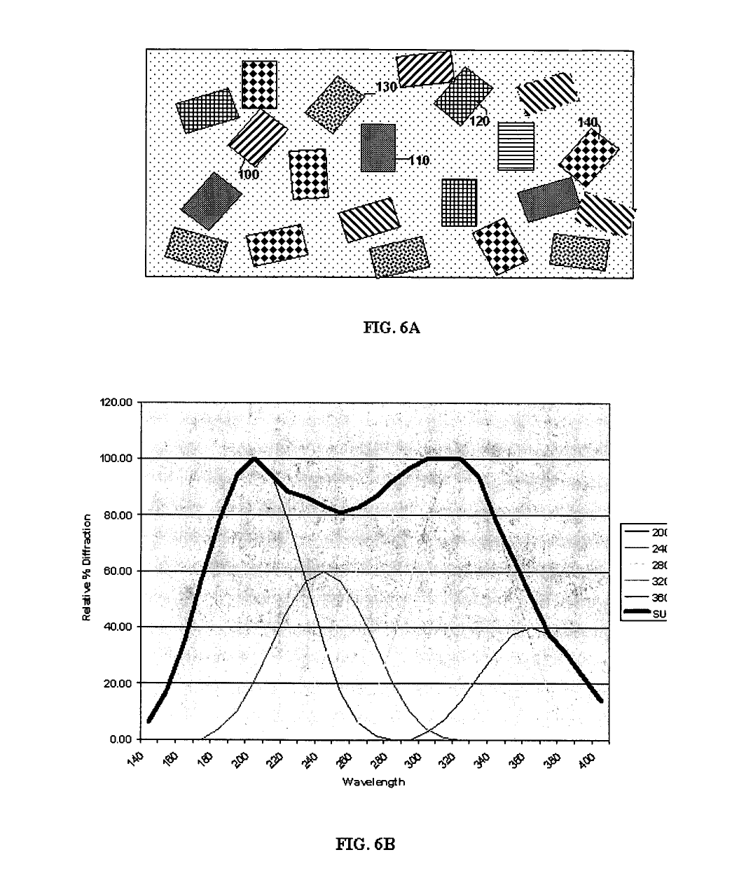

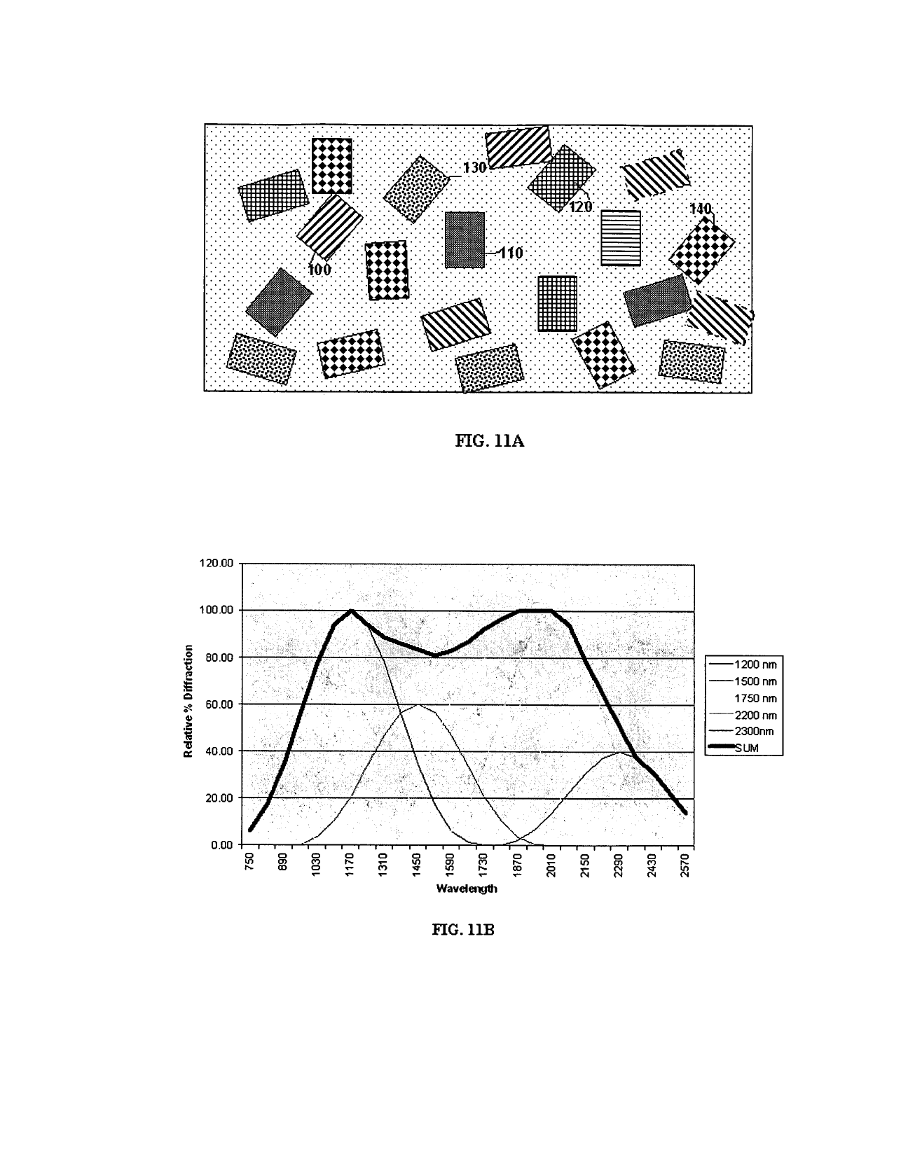

FIGS. 6 and 11 are illustrations of UV and IR blocking compositions that have five different types of crystalline colloidal arrays respectively. Referring to FIG. 6A, the five different arrays 100, 110, 120, 130, 140 are designed to diffract UV radiation centered at approximately 200 nm, 240 nm, 280 nm, 320, and 360 nm, respectively. The net effect, as shown in FIG. 6B, is a composition that is capable of blocking UV over a broad bandwidth of radiation but “tuned” to allow variable transmission of UV radiation. This can be achieved, for example, by modifying the relative amounts of the different crystalline colloidal array materials that are each designed to diffract UV radiation around a specific wavelength in the final sunblock composition. In this way, the composition can be designed to provide customized UV protection depending on a person's skin type. For example, individuals that achieve quick tanning from UVA may prefer to use a sunscreen composition having crystalline colloidal array materials designed to minimize broadband UVA radiation diffraction. FIGS. 11A and 11B provide similar illustrations for an IR blocking composition (e.g., there are five different crystalline colloidal arrays 100, 110, 120, 130, 140 that are designed to diffract IR radiation centered at approximately 1200 nm, 1500 nm, 1750 nm, 2220 nm, and 2300 nm, respectively). The IR blocking composition in FIG. 6B is capable of blocking IR over a broad bandwidth of radiation but “tuned” to allow variable transmission of IR radiation.

C. Random Orientation of Crystalline Colloidal Arrays

Most sunblock and sunscreen compositions have a broad range of UV or IR radiation diffraction. In many instances, a single crystalline colloidal array can only provide electromagnetic protection in a narrow range because of its narrow band of radiation diffraction. These types of limited or narrow range composition are contemplated by the inventor. A preferred composition of the present invention, however, is capable of providing a broad range of electromagnetic radiation diffraction.

In order to obtain broadband protection, a plurality of different types of crystalline colloidal arrays can be used. For example, if each crystalline colloidal array blocks about 0.2 to 2 nm of UV radiation, to produce a sunblock composition that provided broad UV protection (e.g. 200 nm UV spectrum), then the sunblock composition should include approximately 100 to 1000 different crystalline colloidal array materials. This can become expensive and unyielding for manufacture.

The inventor has discovered that crystalline colloidal arrays that predominantly orient randomly increases the range of diffraction of electromagnetic radiation for a given array (see, e.g., FIGS. 3 and 8). This random orientation can be exploited to produce crystalline colloidal array materials that produce broadband electromagnetic radiation protection without the need for a large number of different crystalline colloidal arrays. Reducing the number of different arrays in a given composition can be advantageous for several reasons, including the costs associated with preparing such a composition. Additionally, reducing the number of materials in a composition can benefit the effectiveness and tactile properties of a composition.

Therefore, a non-limiting aspect of the present invention includes designing crystalline colloidal arrays that can align randomly in sunblock compositions. For instance, at least one of the different crystalline colloidal arrays randomly orient in the composition. In other embodiments, at least two, three, four, five, six, seven, or more or all of the different crystalline arrays randomly orient in the composition.

D. Source of Compounds, Agents, and Active Ingredients

The compounds, agents, and active ingredients (e.g., crystal colloidal arrays, particles or matrices of such arrays and their corresponding components, nanoparticles, and other compounds, agents, and active ingredients described herein) that are described in the claims and specification can be obtained by any means known to a person of ordinary skill in the art. In a non-limiting embodiment, for example, the compounds, agents, and active ingredients can be isolated by obtaining the source of such compounds, agents, and active ingredients. In many instances, the compounds, agents, and active ingredients are commercially available. For example, crystalline colloidal arrays can be purchased through PPG Industries Ohio, Inc.

E. Modifications and Derivatives

Modifications or derivatives of the compounds, agents, and active ingredients disclosed throughout this specification are contemplated as being useful with the methods and compositions of the present invention. Derivatives may be prepared and the properties of such derivatives may be assayed for their desired properties by any method known to those of skill in the art.

In certain aspects, “derivative” refers to a chemically modified compound that still retains the desired effects of the compound prior to the chemical modification. Such derivatives may have the addition, removal, or substitution of one or more chemical moieties on the parent molecule. Non limiting examples of the types modifications that can be made to the compounds and structures disclosed throughout this document include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, hetero atoms such as N, S, or O can be substituted into the structure instead of a carbon atom.

F. Equivalents

Known and unknown equivalents to the specific compounds, agents, and active ingredients discussed throughout this specification can be used with the compositions and methods of the present invention. The equivalents can be used as substitutes for the specific compounds, agents, and active components. The equivalents can also be used to add to the methods and compositions of the present invention. A person of ordinary skill in the art would be able to recognize and identify acceptable known and unknown equivalents to the specific compounds, agents, and active ingredients without undue experimentation.

G. Compositions of the Present Invention

A person of ordinary skill would recognize that the compositions of the present invention can include any number of combinations of compounds, agents, and/or active ingredients, or derivatives therein. It is also contemplated that that the concentrations of the compounds, agents, and/or active ingredients can vary. In non-limiting embodiments, for example, the compositions may include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the compounds, agents, active ingredients, or derivatives that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of the compounds, agents, or active ingredients, to the disclosed methods and compositions.

The disclosed compositions of the present invention may also include various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

H. Vehicles

The compositions of the present invention can be incorporated into all types of are effective in all types of vehicles. Non-limiting examples of suitable vehicles include emulsions (e.g., water-in-oil, water-in-oil-in-water, oil-in-water, -oil-in-water-in-oil, oil-in-water-in-silicone emulsions), creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention. In certain aspects, it is important that the concentrations and combinations of the compounds, ingredients, and active agents be selected in such a way that the combinations are chemically compatible and do not form complexes which precipitate from the finished product.

I. Cosmetic Products and Articles of Manufacture

The composition of the present invention can also be used in many cosmetic products including, but not limited to, sunscreen products, sunless skin tanning products, hair products, finger nail products, moisturizing creams, skin benefit creams and lotions, softeners, day lotions, gels, ointments, foundations, night creams, lipsticks, cleansers, toners, masks, or other known cosmetic products or applications. Additionally, the cosmetic products can be formulated as leave-on or rinse-off products.

The compositions or crystalline colloidal arrays of the invention can be used to provide protection from electromagnetic radiation in non-cosmetics applications and products. By way of example only, a series of different crystalline colloidal arrays can be dispersed into a polymeric medium such as paint, ink, or other polymeric pigment vehicle. Additives can be mixed with the pigment vehicle to achieve the final desired effects. These additives can include, in non-limiting aspects, lamellar pigments (e.g., aluminum flakes, graphite, carbon aluminum flakes, mica flakes, and the like) or non-lamellar pigments (e.g., aluminum powder, carbon black, and other organic and inorganic pigments such as titanium dioxide, and the like).

Non-limiting examples of the different types of articles of manufacture and products that the compositions and crystalline colloidal arrays can be used with include protective clothing (e.g., IR protective clothing to deflect or reduce the warm feeling associated with IR radiation), eye glasses, coatings for windows, windows, plastics, wood, stains, and coatings.

J. Additional Compounds and Agents that can be Used in Combination with the Present Compositions

Compositions of the present invention can include other beneficial agents and compounds such as, for example, sun blocking agents, acute or chronic moisturizing agents (including, e.g., humectants, occlusive agents, and agents that affect the natural moisturization mechanisms of the skin), anti-oxidants, sunscreens having UVA and/or UVB protection, emollients, anti-irritants, vitamins, trace metals, anti-microbial agents, botanical extracts, fragrances, dyes and color ingredients, structuring agents, thickening Agent (thickeners and gelling agents), and/or emulsifiers (see U.S. Pat. No. 6,290,938).

1. Sunblock Agents

Sunblock agents that can be used in combination with the compositions and crystalline colloidal arrays of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (and octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate), anthranilates, ethyl urocanate, homosalate, and Parsol 1789. Non-limiting examples of physical sunblocks include kaolin, talc and metal oxides (e.g., titanium dioxide and zinc oxide).

2. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (asminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquatemium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

3. Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

4. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

5. Thickening Agents (Including Thickeners and Gelling Agents)

In certain embodiments, the compositions of the present invention can include one or more thickening agents. Nonlimiting examples include carboxylic acid polymers, crosslinked polyacrylate polymers, polyacrylamide polymers, polysaccharides, and gums.

Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80. Examples of commercially available carboxylic acid polymers include carbomers, which are homopolymers of acrylic acid crosslinked with allyl ethers of sucrose or pentaerytritol (e.g., Carbopol™ 900 series from B.F. Goodrich.

Examples of crosslinked polyacrylate polymers include cationic and nonionic polymers. Examples are described in U.S. Pat. Nos. 5,100,660; 4,849,484; 4,835,206; 4,628,078; 4,599,379).

Examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Other useful polysaccharides include scleroglucans comprising a linear chain of (1-3) linked glucose units with a (1-6) linked glucose every three unit.

Examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

6. Emulsifiers

The compositions of the present invention can also comprise one or more emulsifiers. Emulsifiers can reduce the in interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

7. Additional Compounds and Agents

Non-limiting examples of additional compounds and agents that can be used with the compositions of the present invention include, vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), emollients (i.e. organic esters, fatty acids, lanolin and its derivatives, plant and animal oils and fats, and di- and triglycerides), antimicrobial agents (e.g., triclosan and ethanol), and fragrances (natural and artificial).

K. Kits

In further embodiments of the invention, there is a provided a kit. Any of the compositions, compounds, agents, or active ingredients described in this specification may be comprised in a kit. In a non-limiting example, a kit can include a sunscreen composition, a cosmetic product, or other products and articles of manufacture.

Containers of the kits can include a bottle, dispenser, package, compartment, or other types of containers, into which a component may be placed. The containers can dispense a pre-determined amount of the component (e.g. compositions of the present invention). The composition can be dispensed in a spray, an aerosol, or in a liquid form or semi-solid form. The containers can have spray, pump, or squeeze mechanisms. The container can include indicia on its surface. The indicia, for example, can be a word, a phrase, an abbreviation, a picture, or a symbol. The word or phrase can be “sunscreen,” “sunblock,” “UV specific sunblock,” ext.

Where there is more than one component in the kit (they may be packaged together), the kit also will generally contain a second, third or other additional containers into which the additional components may be separately placed. The kits of the present invention also can include a container housing the components in close confinement for commercial sale. Such containers may include injection or blow-molded plastic containers into which the desired bottles, dispensers, or packages are retained.

A kit can also include instructions for employing the kit components as well the use of any other compositions, compounds, agents, active ingredients, or objects not included in the kit. Instructions may include variations that can be implemented. The instructions can include an explanation of how to apply, use, and maintain the products or compositions, for example.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Preparing an IR or UV Radiation Diffracting Crystalline Colloidal Array

Step 1—Organic Polymer Matrix: The first step in making a UV or IR sunblock array is to prepare an organic polymer matrix. One example of an organic polymer matrix is an ultraviolet radiation curable organic composition. A description of preparing such a matrix is described in U.S. Pat. No. 6,894,086. This process includes: Diphenyl(2,4,6-trimethylbenzoyl)phosphine oxide/2-hydroxy-2-methylpropiophenone (40 grams), 50/50 blend from Aldrich Chemical Company, Inc., Milwaukee, Wis., in 116 g of ethyl alcohol and 250 g of ethoxylated (4) pentaerythritol tetraacrylate, from Sartomer Company, Inc., Exton, Pa., were added with stirring to 750 g neopentyl glycol diacrylate from Sartomer Company, Inc., Exton, Pa. Zinc oxide nanopowder (50-70 nm) (Aldrich Chemical Company, Inc.) is added at varying amounts (0 g to 2.5 g) depending on the final refractive index that is desired.

Step 2—Particles: Organic or inorganic particles can be used. A description of preparing a polystyrene particle that can be used with the present invention is described in U.S. Pat. No. 6,894,086. Polystyrene particles in water (average diameter 0.1 μm, 10% in water) can be obtained from Aldrich Chemical Company, Inc., Milwaukee, Wis. The particles were dialyzed in regenerated cellulose dialysis tubing (Fisher Scientific, Pittsburgh, Pa.) against deionized water for approximately 500 hours. The deionized water was exchanged on average every 30 hours. Ultrafiltrate is then removed until the solids content of the mixture was 40 percent by weight.

Step 3—Film formation: Making films that include a crystalline colloidal array is described in U.S. Pat. No. 6,894,086. One example is to take 700 g of polystyrene-divinylbenzene silica particles prepared in Step 2 and apply, via slot-die coater (Frontier Technologies, Towanda, Pa.) to a polyethylene terephthalate substrate. This silica particle coated substrate is then dried at 150° F. for 1 minute to a porous dry film thickness of approximately 2.5 microns. The organic polymer matrix material, 100 grams, prepared from Step 1 is then applied via slot-die coater into the interstitial spaces of the porous dry film on the polyethylene terephthalate substrate. The coated film is then dried at 120° F. for 1 minute, and then ultraviolet radiation cured using a 100 W mercury lamp. The hardened film was then removed from the polyethylene terephthalate substrate.

Step 4—Crystalline Colloidal Array Formation: The resulting hardened films are made into coarse particles and then reduced to fine, uniform particles with aspect ratios less than 2:1. Placing the film in a blender can produce coarse particles. Fine particles can be produced (<5 microns) by using fluid energy mills such as “MicronMaster,” “Majac,” “Jet-O-Mizers” mills, and other suitable mills for fine grinding. An especially useful fluid energy mills is the “Jet-O-Mizer” made by Fluid Energy Process and Equipment Company, Hatfield, Pa.

The resulting colloidal arrays can diffract UV or IR radiation at about 300 nm and 1200 nm, respectively. Changing the concentration of zinc oxide nanopowder in the polymer matrix results in a shift in the UV or IR radiation diffracted. As a result, a series of UV or IR Sunblock Agents can be made by simply modifying the concentration of zinc oxide. This series can be combined in a composition to give a tunable, broad wavelength sunblock composition.

### Example 2

### Non-Limiting Example of a Sunscreen Composition

A non-limiting example of sunscreen composition of the present invention is described in Table 1 below. The ingredients in Table 1 was formulated for topical application to human skin.

TABLE 1
Sunscreen Composition*

| Ingredient | Weight % |
|---|---|
| Crystalline Colloidal Arrays | 5% |
| Phenylbenzimidazole | 1.5 |
| Sulfonic Acid | |
| Isopropyl Palmitate | 8.0 |
| Butylene Glycol | 2.0 |
| Triethanolamine | 1.6 |
| Glycerin | 1.0 |
| Stearic Acid | 1.0 |
| Cetyl Alcohol | 0.75 |
| DEA Cetyl Phosphate | 0.75 |
| PVP Eicosene Copolymer | 0.5 |
| Stearyl Alcohol | 0.25 |
| Methylparaben | 0.25 |
| Carbomer 954 | 0.2 |
| Propylparaben | 0.15 |
| Acrylates/C₁₀-C₃₀ | 0.125 |
| Alkyl crylate | |
| Crosspolymer | |
| Disodium EDTA | 0.1 |
| Water | q.s. |
| *Prepare the water phase by mixing in a suitable vessel, the Carbomer 954 and the acrylates/C₁₀-C₃₀ alkyl | |
| # acrylates crosspolymer in all but 4% of the water. Add the butylene glycol, glycerin, disodium EDTA, and methylparaben to the | |
| # water phase and heat to 80° C. Prepare the oil phase in a separate vessel by mixing the isopropyl palmitate,crystalline colloidal arrays, propylparaben, DEA cetyl phosphate, stearic acid, | |
| # cetyl alcohol, stearyl alcohol, and PVP eiscosene copolymer and heating to 80° C. When both phases reach 80° C., slowly add the oil phase to the water phase while | |
| # milling the system to form an emulsion. Cool the system under agitation. Once the system reaches 70° C., add a premix | |
| # containing 0.73% of the triethanolamine and 1% of the water to the batch. When the batch cools to about 45° C., add a | |
| # premix containing the phenylbenzimidazole sulfonic acid, remaining triethanolamine, and remaining water to the batch, cool to 30° C. and pour into suitable containers. | |

As noted above, the sunscreen composition in Table 1 is a non-limiting example. Additionally, it is contemplated that derivatives of the ingredients in Table 1 can be used as substitutes, additional ingredients can be added and/or deleted from the sunscreen composition described in Table 1. These and other aspects of the present invention are disclosed throughout this specification.

All of the compositions and/or methods disclosed and claimed in this specification can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 2,798,053
U.S. Pat. No. 3,755,560
U.S. Pat. No. 4,421,769
U.S. Pat. No. 4,434,010
U.S. Pat. No. 4,509,949
U.S. Pat. No. 4,599,379
U.S. Pat. No. 4,628,078
U.S. Pat. No. 4,828,825
U.S. Pat. No. 4,835,206
U.S. Pat. No. 4,849,484
U.S. Pat. No. 5,011,681
U.S. Pat. No. 5,059,245
U.S. Pat. No. 5,087,445
U.S. Pat. No. 5,100,660
U.S. Pat. No. 5,171,363
U.S. Pat. No. 5,427,771
U.S. Pat. No. 6,290,938
U.S. Pat. No. 6,894,086
CTFA International Cosmetic Ingredient Dictionary, Fourth edition, 1991, pp. 12 and 80.
Chel V G M et al., Ultraviolet irradiation corrects vitamin D deficiency and suppresses secondary hyperparathyroidism in the elderly, Journal of Bone and Mineral Research, August 1998, Volume 13, Number 8, Page 1238.
Holick M F, Sunlight and vitamin D for bone health and prevention of autoimmune diseases, cancers, and cardiovascular disease, American Journal of Clinical Nutrition, Vol. 80, No. 6, 1678S-1688S, December 2004.
Horwitz L R et al., Augmentation of wound healing using monochromatic infrared energy, Adv Wound Care. 1999 Jan.-Feb.; 12(1):35-40.).
McCutcheon's, Detergents and Emulsifiers, North American Edition (1986).
Tangpricha V, Turner A, et al. Tanning is associated with optimal vitamin D status (serum 25-hydroxyvitamin D concentration) and higher bone mineral density by, American Journal of Clinical Nutrition, Vol. 80, No. 6, 1645-1649, December 2004.
Sayre, R. M et al., Physical Sunscreens, J. Soc. Cosmet. Chem., vol. 41, no. 2, pp. 103-109 1990.
U.S. Sunscreen Tentative Final Monograph, issued in May, 1993.

1. A composition comprising a plurality of different crystalline colloidal arrays, the arrays comprising particles dispersed within a matrix, wherein at least one of the different crystalline colloidal arrays randomly orientates within the composition, and wherein the composition diffracts electromagnetic radiation.

2. The composition of claim 1 comprising:
(a) a first crystalline colloidal array that diffracts electromagnetic radiation over a selected wavelength range; and
(b) a second crystalline colloidal array that diffracts electromagnetic radiation over a selected wavelength range that is different than the first colloidal array.

3. The composition of claim 2, wherein the composition includes a third crystalline colloidal array comprising particles dispersed within a third matrix, wherein the third colloidal array diffracts electromagnetic radiation over a selected wavelength range that is different than the first and second colloidal arrays.

4. (canceled)

5. The composition of claim 1, wherein the composition includes up to 15 to 100 different crystalline colloidal arrays that each diffract electromagnetic radiation over different selected wavelength ranges.

6. (canceled)

7. The composition of claim 1, wherein at least one of the different crystalline colloidal arrays has an aspect ratio equal to or less than 2:1.

8. The composition of claim 1, wherein at least one of the different crystalline colloidal arrays has an aspect ratio equal to or greater than 2:1.

9. The composition of claim 1, wherein all of the different crystalline colloidal arrays randomly orientate within the composition.

10. The composition of claim 1, wherein the composition diffracts a broad spectrum of electromagnetic radiation.

11. The composition of claim 1, wherein the particles in one of the crystalline colloidal arrays are organized into a periodic array.

12-36. (canceled)

37. The composition of claim 1, wherein the composition diffracts UV radiation.

38. The composition of claim 37, wherein the composition selectively diffracts a predetermined wavelength range of UV radiation.

39. The composition of claim 38, wherein the predetermined wavelength is about 200 to about 400 nm.

40. The composition of claim 39, wherein the predetermined wavelength is about 200 to about 290 nm.

41. The composition of claim 39, wherein the predetermined wavelength is about 290 to about 320.

42. The composition of claim 37, wherein the composition permits transmission of a predetermined wavelength range of UV radiation.

43. The composition of claim 42, wherein the composition does not diffract UV radiation having a wavelength of about 321 to about 400 nm.

44. The composition of claim 42, wherein the composition does not diffract UV radiation having a wavelength of about 290 to about 315 nm.

45. The composition of claim 44, wherein the composition does not diffract UV radiation having a wavelength of about 309 to about 314 nm.

46. The composition of claim 1, wherein the composition diffracts IR radiation.

47. The composition of claim 46, wherein the composition selectively diffracts a predetermined wavelength range of IR radiation.

48. The composition of claim 47, wherein the predetermined wavelength is about 760 to about 2,500 nm.

49. The composition of claim 46, wherein the composition permits transmission of a predetermined wavelength range of IR radiation.

50. The composition of claim 48, wherein the composition does not diffract IR radiation having a wavelength of about 1660 to about 1900 nm.

51. The composition of claim 1, wherein the composition is transparent.

52-54. (canceled)

55. The composition of claim 1, wherein the composition is comprised in a vehicle.

56. The composition of claim 55, wherein the vehicle comprises an emulsion, a cream, a lotion, a solution, an anhydrous base, a gel, a spray, or an ointment.

57. (canceled)

58. The composition of claim 1, wherein the composition is comprised in a product.

59. The composition of claim 58, wherein the product is a skin sunscreen product, a skin care product, paint, ink, a glass coating, glass, cloth, plastic, or eye glasses.

60-71. (canceled)

72. A sunscreen composition comprising a plurality of different crystalline colloidal arrays, the arrays comprising particles dispersed within a matrix, wherein the composition is formulated to be applied to skin and diffracts electromagnetic radiation.

73. The sunscreen composition of claim 72 comprising:
(a) a first crystalline colloidal array that diffracts electromagnetic radiation over a selected wavelength range; and
(b) a second crystalline colloidal array that diffracts electromagnetic radiation over a selected wavelength range that is different than the first colloidal array.

74. The composition of claim 72, wherein at least one of the different crystalline colloidal arrays has an aspect ratio equal to or less than 2:1.

75. The composition of claim 72, wherein at least one of the different crystalline colloidal arrays has an aspect ratio equal to or greater than 2:1.

76. The composition of claim 72, wherein at least one of the different crystalline colloidal arrays randomly orientate within the composition.

77. The composition of claim 76, wherein all of the different crystalline colloidal arrays randomly orientate within the composition.

78. The sunscreen composition of claim 72, wherein the composition is transparent.

79. The sunscreen composition of claim 72, wherein the composition is formulated to be spread or sprayed onto the skin.

80. The sunscreen composition of claim 72, wherein the composition is comprised in a vehicle.

81. The sunscreen composition of claim 80, wherein the vehicle comprises an emulsion, a cream, a lotion, a solution, an anhydrous base, a gel, a spray, or an ointment.

82-89. (canceled)

90. A method of protecting an object from electromagnetic radiation comprising applying on the surface of the object or in incorporating into the object the composition of claims 1 or 72.

91. The method of claim 90, wherein the composition is topically applied to the object.

92. The method of claim 91, wherein the object is skin.

93-95. (canceled)

96. The method of claim 90, wherein the composition is incorporated into the object.

97. The method of claim 96, wherein the object is paint or ink.

98-121. (canceled)
